(19) United States
(12) Patent Application Publication
(10) Pub. No.: US 2017/0022125 A1
(43) Pub. Date: Jan. 26, 2017

(54) PROCESSES FOR PRODUCING POLYMER GRADE LIGHT OLEFINS FROM MIXED ALCOHOLS

(71) Applicant: UOP LLC, Des Plaines, IL (US)

(72) Inventor: Geoffrey W. Fichtl, Chicago, IL (US)

(21) Appl. No.: 15/214,835

(22) Filed: Jul. 20, 2016

Related U.S. Application Data
(60) Provisional application No. 62/195,077, filed on Jul. 21, 2015.

(51) Int. Cl. C07C 1/24; C07C 7/12; C07C 6/04; C07C 4/02; C07C 5/25; C07C 41/06; C07C 1/22; C07C 7/00; C07C 7/09

(52) U.S. Cl. CPC C07C 1/24; C07C 7/005; C07C 7/12; C07C 6/04; C07C 7/09; C07C 5/2506; C07C 41/06; C07C 1/22; C07C 4/02; C07C 2521/12; C07C 2521/08; C07C 2521/04

(57) ABSTRACT

Processes for providing a high purity olefin product are described. The processes involve dehydrating a feedstream comprising a mixture of alcohols having 3 to 8 carbon atoms and forming a mixed olefin stream and a water stream, the mixed olefin stream comprising a mixture of olefins having 3 to 8 carbon atoms. The mixed olefin stream is separated into at least a C₃ olefin stream comprising olefins having 3 carbon atoms and a C₄₋₈ olefin stream comprising olefins having 4 to 8 carbon atoms. The C₄₋₈ olefin stream is separated into a C₄ olefin stream comprising olefins having 4 carbon atoms and a C₅₋₈ olefin stream comprising olefins having 5 to 8 carbon atoms. At least one of the C₃ olefin stream and the C₄ olefin stream is purified.

### CROSS-REFERENCE TO RELATED APPLICATION

This application claims priority from Provisional Application No. 62/195,077 filed Jul. 21, 2015, the contents of which are hereby incorporated by reference.

### BACKGROUND OF THE INVENTION

Because the demand for light olefins is increasing globally, interest in alternative sources of high purity propylene and butenes will increase.

Propylene is used as an intermediate to produce a multitude of petrochemicals, chiefly polypropylene. Polymer grade propylene is a high purity product (99.9 wt%) and is typically made by thermally cracking naphtha or as a byproduct of gasoline production via fluid catalytic cracking. Other methods for production of propylene include propane dehydrogenation, metathesis of ethylene with higher olefins, and conversion of methanol to olefins.

Butene, more specifically, isobutylene is used as an intermediary to produce a variety of chemicals including polymers and gasoline oxygenates. Polymer grade isobutylene comprises a high purity product (99.9 wt %) and is typically made by dehydrating tert-butyl alcohol or dehydrogenation of isobutane.

Alternative feed sources for propylene and butenes are desirable. Petroleum based feedstocks have become less desirable recently due to concerns about limited petroleum resources, increasing energy demand, greenhouse gas emissions and related climate change concerns.

Therefore, there is a need for an effective and efficient process for the production of polymer grade light olefins.

### SUMMARY OF THE INVENTION

One aspect of the invention is a process for providing a high purity olefin product. In one embodiment, the process involves dehydrating a feedstream comprising a mixture of alcohols having 3 to 8 carbon atoms and forming a mixed olefin stream and a water stream, the mixed olefin stream comprising a mixture of olefins having 3 to 8 carbon atoms. The mixed olefin stream is separated into at least a C₃ olefin stream comprising olefins having 3 carbon atoms and a C₄₋₈ olefin stream comprising olefins having 4 to 8 carbon atoms. The C₄₋₈ olefin stream is separated into a C₄ olefin stream comprising olefins having 4 carbon atoms and a C₅₋₈ olefin stream comprising olefins having 5 to 8 carbon atoms. At least one of the C₃ olefin stream and the C₄ olefin stream is purified.

In some embodiment, the C₃ olefin stream can be purified by separating propane from the C₃ olefin stream.

In some embodiment, the C₄ olefin stream can be purified by separating the C₄ olefin stream into at least an isobutylene stream and a normal butenes stream using a shape selective adsorbent.

In some embodiments, the C₄ olefin stream can be purified by fractionating the C₄ olefin stream into an overhead stream comprising isobutylene and 1-butene and a bottoms stream comprising 2-butene, and separating the overhead stream into an isobutylene stream and a 1-butene stream using a shape selective adsorbent.

In some embodiments, the C₄ olefin stream can be purified by isomerizing the C₄ olefin stream to convert 1-butene to 2-butene; and fractionating the isomerized C₄ olefin stream into an isobutylene stream and a 2-butene stream.

In some embodiments, the C₄ olefin stream can be purified by converting the isobutylene and methanol to methyl tert-butyl ether in an etherification reaction zone to form an etherification effluent; separating the etherification effluent into a methyl tert-butyl ether stream and a stream comprising 1-butene and 2-butene; converting the methyl tert-butyl ether into isobutylene and methanol in a decomposition zone to form a decomposition effluent; and separating the decomposition effluent into an isobutylene stream and a methanol stream. In some embodiments, the stream comprising 1-butene and 2-butene can be fractionated into a 1-butene stream and a 2-butene stream.

In some embodiments, the C₄ olefin stream can be purified a 2-butene stream which is then introduced with an ethylene stream to a metathesis reaction zone to produce a propylene stream.

In some embodiments, the C₅₋₈ olefin stream can be separated into a C₅ olefin stream comprising olefins having 5 carbon atoms and a C₆₋₈ olefin stream comprising olefins having 6 to 8 carbon atoms. In some embodiments, the C₆₋₈ olefin stream can be reformed to form an aromatics stream.

In some embodiments, the C₄₋₈ olefin stream can be cracked in an olefins cracking zone to produce a cracked propylene stream and a cracked C₄₋₈ mixed stream comprising olefins, paraffins and C₆₋₈ aromatics.

### BRIEF DESCRIPTION OF THE DRAWINGS

FIG. 1 illustrates one embodiment of a process for producing polymer grade light olefins according to the present invention.

FIG. 2 illustrates one embodiment of a process for separating a mixture of butenes.

FIG. 3 illustrates another embodiment of a process for separating a mixture of butenes.

FIG. 4 illustrates another embodiment of a process for separating a mixture of butenes.

FIG. 5 illustrates yet another embodiment of a process for separating a mixture of butenes.

FIG. 6 illustrates another embodiment of a process for producing polymer grade light olefins according to the present invention.

FIG. 7 illustrates another embodiment of a process for producing polymer grade light olefins according to the present invention.

### DETAILED DESCRIPTION OF THE INVENTION

Processes for producing polymer grade light olefins from mixed alcohols have been developed. The processes involve producing light olefins via alcohol dehydration. The light olefins generally correspond the feed alcohols in carbon number and carbon atom skeletal arrangement. For example, dehydration of 1-propanol or 2-propanol results in selective production of propylene. Similarly, dehydration of isobutanol results in greater than 85% selective production of isobutylene.

Commercial scale production of C₃ to C₈ mixed alcohols from coal or natural gas-based syngas is expected to increase in the next few years. This would provide an excellent source of mixed alcohols for conversion to polymer grade olefins via relatively simple and low-capital dehydration processes.

Dehydration of mixed alcohols can provide a relatively simple approach to producing high purity light olefins suitable for preparing high value polymer grade olefins.

Suitable dehydration catalysts include homogeneous or heterogeneous catalysts. A nonlimiting list of homogeneous acid catalysts includes: inorganic acids, such as sulfuric acid, hydrogen fluoride, fluorosulfonic acid, phosphotungstic acid, phosphomolybdic acid, and phosphoric acid; Lewis acids such as aluminum and boron halides (e.g., AlCl₃, BF₃, etc.); organic sulfonic acids, such as trifluoromethanesulfonic acid, p-toluenesulfonic acid and benzenesulfonic acid; heteropolyacids; fluoroalkyl sulfonic acids; metal sulfonates; metal trifluoroacetates; compounds thereof, and combinations thereof. A non-limiting list of heterogeneous acid catalysts includes: heterogeneous heteropolyacids (HPAs); solid phosphoric acid; natural clay minerals, such as those containing alumina or silica; cation exchange resins, such as sulfonated polystyrene ion exchange resins; metal oxides, such as hydrous zirconium oxide, Fe₂O₃, Mn₂O₃, γ-alumina, etc.; mixed metal oxides, such as sulfated zirconia/y-alumina, alumina/magnesium oxide, etc.; metal salts, such as metal sulfides, metal sulfates, metal sulfonates, metal nitrates, metal phosphates, metal phosphonates, metal molybdates, metal tungstates, metal borates; zeolites, such as NaY zeolite, H-ZSM-5, NaA zeolite, etc.; modified versions of any of the above known in the art, and combinations of any of the above.

The dehydration reaction is typically carried out using one or more fixed-bed reactors using any of the dehydration catalysts described herein. Alternatively, other types of reactors known in the art can be used, such as fluidized bed reactors, batch reactors, catalytic distillation reactors, etc. In some embodiments, the dehydration catalyst is a heterogeneous acidic γ-alumina catalyst. In some embodiments, the dehydration reaction is carried out in the vapor phase to facilitate removal of water (either present in the dehydration feedstock or as a by-product of the dehydration reaction). In some embodiments, the dehydration reaction is carried out at a pressure ranging from about 0-2068 kPa(g) (0-300 psig), and at a temperature of about 350° C. or less (e.g., about 300-350° C.).

For example, the mixed alcohol feed is dehydrated into light olefins and water. The water is separated from the olefins via density difference. The light olefins stream will likely still contain small amounts of unconverted alcohols and trace byproduct oxygenates. These can be removed using an adsorbent treatment or other processes, such as solvent extraction. After reduction of the trace oxygenates, the light olefins stream is fractionated into a propylene stream, a butenes stream, and a C₅ to C₈ mixed olefin stream.

After fractionation, it may be necessary for additional purification to meet polymer grade specifications. For example, propane can be removed from the propylene stream using fractionation or an adsorption process. The butenes stream can be separated into various fractions using a variety of processes, including combinations of one or more of fractionation, adsorption, and isomerization. The C₅ to C₈ mixed olefin stream can used for blending in gasoline or it can be further treated.

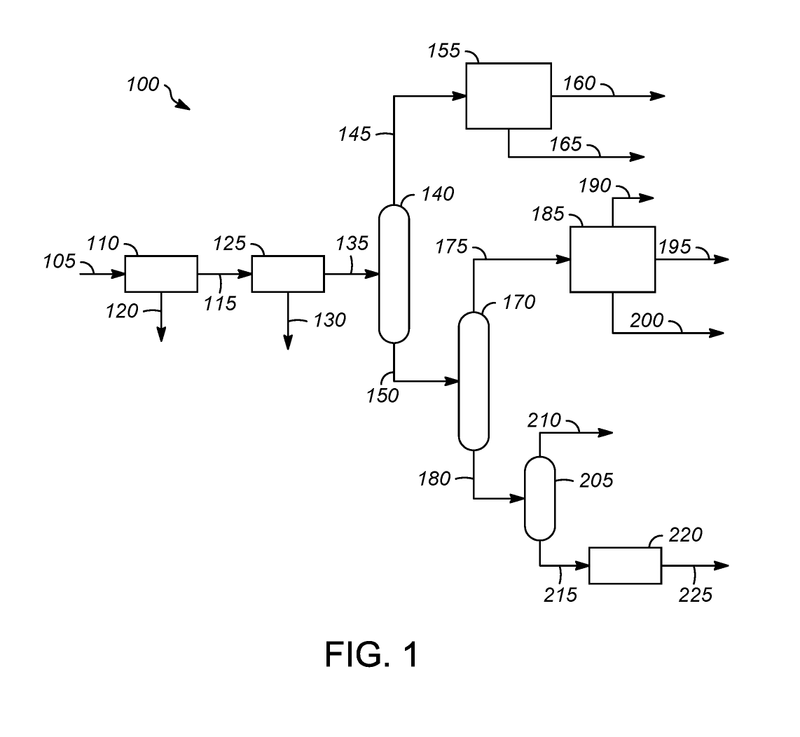

FIG. 1 illustrates a process 100 for producing light olefins from mixed alcohols. The mixed alcohol feed stream 105 contains a mixture of alcohols having 3 to 8 carbon atoms. The mixed alcohol feed stream 105 is sent to a dehydration reaction zone 110. The dehydration zone may comprise one or more suitable catalysts and be operated under sufficient conditions to selectively dehydrate the mixed alcohols and provide a mixed olefin stream 115 and a water stream 120. The mixed olefin stream 115 will comprise a mixture of olefins having 3 to 8 carbon atoms. It will also include oxygenates and other trace components.

The mixed olefin stream 115 is sent to an oxygenate removal zone 125. The oxygenate removal zone 125 may contain various adsorbent beds suitable for removal of trace oxygenates, or may contain an extractive separation process, or another separation process, or a combination of processes, which are capable of separating an oxygenate stream 130 from a purified mixed olefin stream 135.

The purified mixed olefin stream 135 is sent to a first fractionation zone 140 where it is separated into a C₃ olefin stream 145 and a C₄₋₈ olefin stream 150.

The C₃ olefin stream 145 comprises propane as well as propylene. The C₃ olefin stream 145 can be sent to a propylene purification zone 155 to separate the propane from the propylene. The propylene purification zone 155 can utilize fractionation or adsorption, such as an adsorption process using silicalite. The purified propylene stream 160 can be recovered for use as a feed to a polypropylene production unit, or other downstream process for petrochemicals production. The propane stream 165 can be used as a fuel or as feed to a propane dehydrogenation unit to make more propylene.

The C₄₋₈ olefin stream 150 is sent to a second fractionation zone 170 where it is separated into a C₄ olefin stream 175 and a C₅₋₈ olefin stream 180.

The C₄ olefin stream 175 which contains isobutylene, 1-butene, and cis and trans 2-butene, can then be purified in a butene purification zone 185 using various processes as discussed below. The products of the butene purification zone 185 can be an isobutylene stream 190, a 1-butene stream 195, and a 2-butene stream 200.

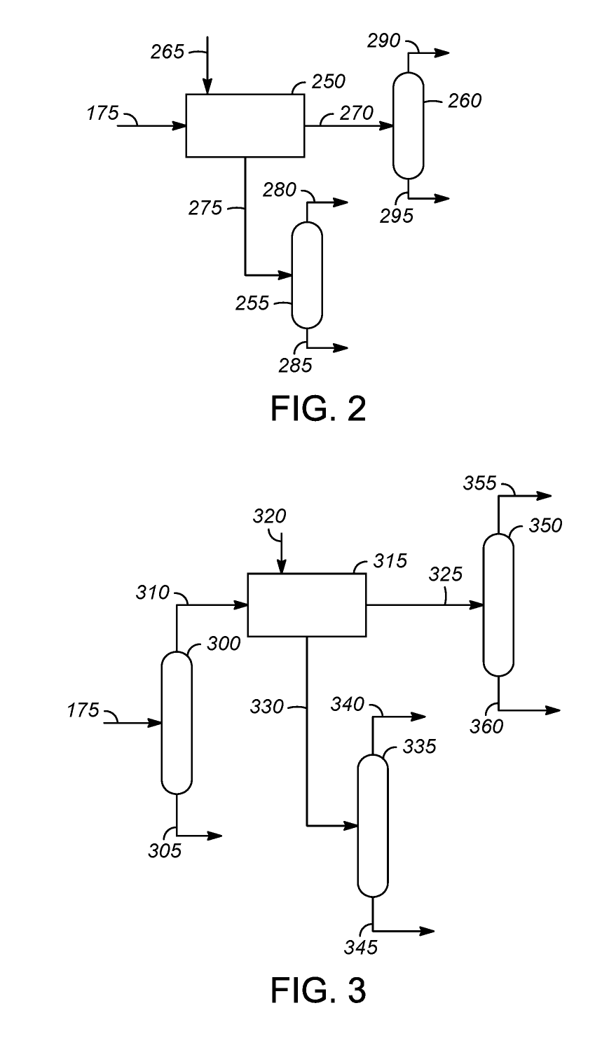

FIG. 2 illustrates one embodiment of a butene purification zone 185 which includes an adsorptive separation zone 250 and at least two fractionation columns 255, 260 downstream from the adsorptive separation zone 250.

The C₄ olefin stream 175 is introduced into the adsorptive separation zone 250 which comprises one or more vessels that include a shape selective adsorbent configured to reject isobutylene and adsorb normal butenes (1-butene, cis-2-butene, and trans-2-butene). A desorbent stream 265 comprising, for example, hexane, could be used to desorb normal butenes from the adsorbent in an extract stream 275. The raffinate stream 270 comprises the isobutylene. Such adsorptive separation zones 250 are known and may comprise a simulated moving bed unit or any other similar unit.

The extract stream 275 may be passed to a first butene fractionation column 255 which is configured to separate the components to provide a mixed normal butene stream 280 and a hexane stream 285. The mixed normal butene stream 280 will include 2-butenes (both cis- and trans-), as well as 1-butene. Although not depicted as such, the mixed normal butene stream 280 may be separated into individual components in another fractionation column or other separation unit. The hexane stream 285 may be recycled as desorbent for the adsorptive separation zone 250.

The raffinate stream 270 from the adsorptive separation zone 250 comprises a mixture of isobutylene and hexane. The raffinate stream 270 is passed to a second butene fractionation column 260 which is configured to separate the components to provide a high purity isobutylene stream 290 and a hexane stream 295 which may be recycled as desorbent to the adsorptive separation zone 250.

FIG. 3 illustrates another embodiment of the butene purification zone 185. In this embodiment, the C₄ olefin stream 175 is separated by the first butene fractionation column 300 into a 2-butene stream (comprising both cis- and trans-) 305 and a stream 310 comprising isobutylene and 1-butene.

From the first butene fractionation column 300, the stream 310 comprising isobutylene and 1-butene is passed to the adsorptive separation zone 315 which, again, may comprise one or more vessels that include a shape selective adsorbent configured to reject isobutylene and adsorb the 1-butene. A desorbent stream 320 comprising, for example, hexane, could be used to desorb 1-butene from the adsorbent in an extract stream 330. The raffinate stream 325 comprises the isobutylene.

The extract stream 330 may be passed to a second butene fractionation column 335 which is configured to separate the components to provide a 1-butene stream 340 and a hexane stream 345. Similar to the previous embodiment, the hexane stream 345 can be recycled as desorbent in the adsorptive separation zone 315.

The raffinate stream 325 from the adsorptive separation zone 315 comprises a mixture of isobutylene and hexane and may be passed to a third butene fractionation column 350 which is configured to separate the components to provide a high purity isobutylene stream 355 and a hexane stream 360 which may be recycled.

In comparison to the embodiment shown in FIG. 2, the embodiment shown in FIG. 3 will provide for a smaller adsorptive separation zone 315 as less material is treated. Additionally, the fractionation before adsorptive separation will provide three separate butene streams: isobutylene stream 355, 1-butene stream 340, and 2-butenes stream 305.

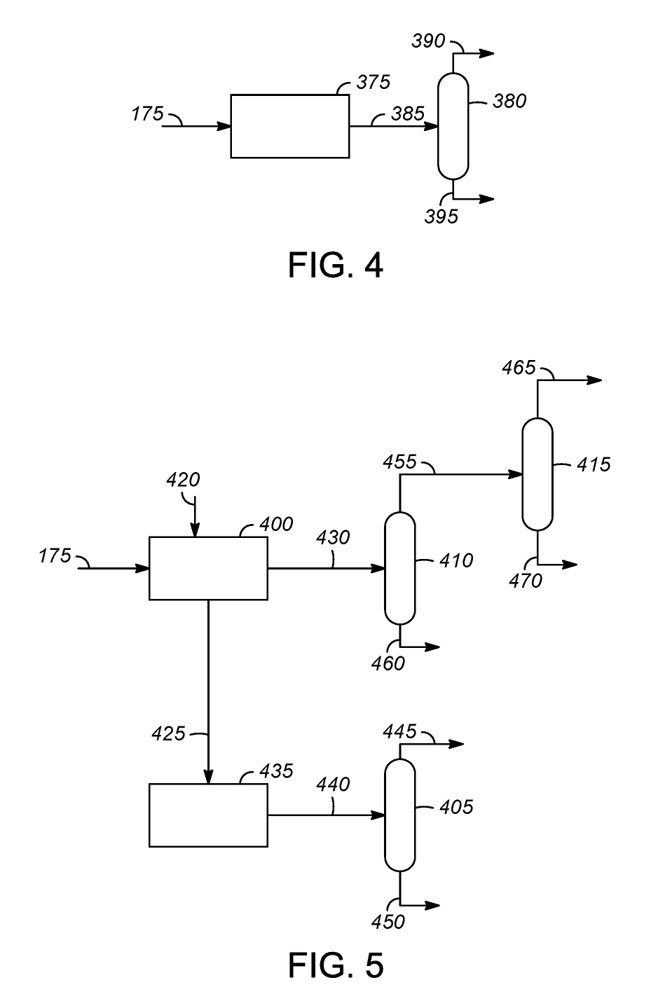

Turning to FIG. 4, in this embodiment, the butene purification zone comprises an isomerization zone 375 and a butene fractionation column 380. As shown, the C₄ olefin stream 175 is passed into an isomerization zone 375 having one or more vessels including a catalysts and being operated under conditions to selectively convert 1-butene into 2-butene. Such processes and conditions are known to those of ordinary skill in the art.

An isomerized effluent 385 from the isomerization zone 375 will comprise isobutylene and 2-butenes. Accordingly, the isomerized effluent 385 may be passed to the butene fractionation column 380 which separates the isomerized effluent 385 into a high purity isobutylene stream 390 and a 2-butene stream 395.

With reference to FIG. 5, in this embodiment, the butene purification zone includes a selective reactive zone 400 and may also include one or more fractionation columns 405, 410, 415.

The selective reaction zone 400 comprises a zone that will convert the isobutylene into one or more intermediary chemicals and separate the intermediary chemicals from the 1-butene and 2-butenes. For example, the selective reaction zone 400 may receive the C₄ olefin stream 175 and a methanol stream 420. It is operated under reaction conditions in the presence of a catalyst to react the isobutylene with the methanol to form methyl tert-butyl ether (MTBE). Such a selective reaction zone 400 may be, for example, an EtherMax® unit from UOP LLC. The selective reaction zone 400 will provide an MTBE stream 425 and a stream 430 comprising 1-butene and 2-butenes.

The MTBE stream 425 may be passed to a decomposition zone 435 configured to promote the decomposition of MTBE into methanol and isobutylene. An effluent stream 440 from the decomposition zone 435 may be passed to a first butene fractionation column 405 to separate a high purity isobutylene stream 445 and a methanol stream 450, which may be recycled to the selective reaction zone 400.

The stream 430 comprising 1-butene and 2-butenes from the selective reaction zone 400 may be passed to a second butene fractionation column 410 to separate an overhead stream 455 from a 2 butene stream 460. The overhead stream 455 may be passed to a third butene fractionation column 415 to separate a light ends stream 465 from a 1-butene stream 470.

Returning to FIG. 1, the C₅₋₈ olefin stream 180 can be sent to a third fractionation zone 205 where it is separated into a C₅ olefin stream 210 and a C₆₋₈ olefin stream 215. The C₅ olefin stream 210 can be sent to the gasoline pool, to a downstream olefin metathesis zone, or to an olefin cracking zone to convert the C₅ olefins into predominantly propylene (not shown). The C₆₋₈ olefin stream 215 can be sent to a reforming zone 220 to produce an aromatics stream 225 which can be sent to an aromatics complex for further treatment (not shown).

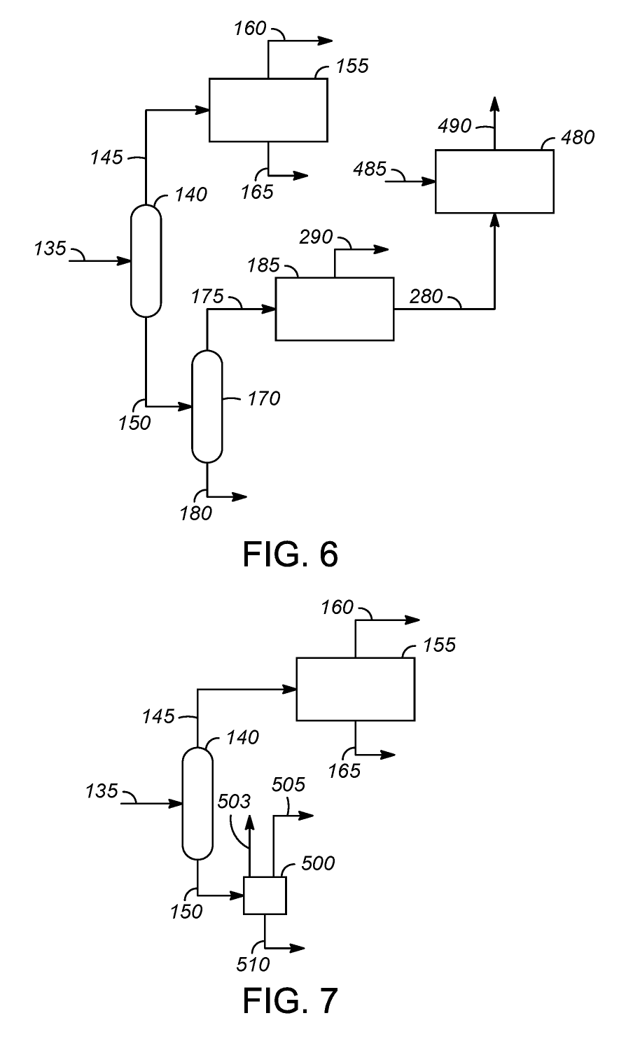

FIG. 6 illustrates a process designed to increase the production of propylene. The butene purification zone 185 produces a 2-butene stream 280 using one of the processes discussed above. The 2-butene stream 280 is sent to an olefin metathesis reaction zone 480 along with ethylene stream 485 to produce propylene stream 490 which can be recovered. In some embodiments, the C₅ olefin stream 210 (from FIG. 1) could be sent to olefin metathesis reaction zone 480 (not shown).

FIG. 7 illustrates another process designed to increase propylene production. In this process, the C₄₋₈ olefin stream 150 is sent to an olefin cracking zone 500 where some of the C₄₋₈ olefins are cracked into C₃ and C₂ olefins. The cracked product is separated into a C₂ olefin steam 503, a C₃ olefin steam 505, and a C₄+ stream 510. The C₂ olefin steam 503 and the C₃ olefin steam 505 can be recovered.

Thus, the recovery of high purity propylene and butene streams from a mixed alcohol stream may be accomplished.

### EXAMPLE 1

A mixed alcohol feed having the feed composition in Table 1 was dehydrated into olefins over an amorphous silica-alumina catalyst. The dehydration was completed at a reactor temperature of about 340.6° C. (645° F.), at a reactor pressure of about 1965 kPa(g) (285 psig), and a weight-hourly-space velocity of about 5.0 hr⁻¹. The resulting non-water product distribution is shown in Table 2. The data shows that significant amounts of oxygenates and unconverted alcohols remained in the non-water reactor effluent after dehydration. Operation at higher conversion and proper removal of trace oxygenates would have reduced these byproducts to more manageable levels, in which case, the product would be suitable feed to the olefins separation process described herein.

TABLE 1
Mixed Alcohol Feed Compositions

| | | |
|---|---|---|
| 2-Propanol | mass % | 0.3 |
| 1-Butanol | mass % | 4.8 |
| 1-Propanol | mass % | 26.9 |
| 2-Methyl-1-butanol | mass % | 5.2 |
| 1-Pentanol | mass % | 6.3 |
| 1-Hexanol | mass % | 4.5 |
| 2-Butanol | mass % | 1.6 |
| 2-Ethyl-1-hexanol | mass % | 1.0 |
| 1-Heptanol | mass % | 1.6 |
| 2-methyl-1-propanol | mass % | 47.8 |

TABLE 2
Amorphous Silica Alumina - Normalized for Water

| | | |
|---|---|---|
| Paraffins | mass % | 2.2 |
| Ethylene | mass % | 0.0 |
| Propylene | mass % | 19.6 |
| 1-Butene&I-Butylene | mass % | 29.8 |
| Cis-2-butene | mass % | 7.1 |
| Trans-2-butene | mass % | 10.0 |
| Pentenes | mass % | 11.6 |
| Hexenes | mass % | 4.8 |
| Heptenes | mass % | 0.0 |
| C8 Olefins | mass % | 6.5 |
| C12 Olefins | mass % | 0.7 |
| Other Hydrocarbons | mass % | 0.1 |
| non-alcohol oxygenates | mass % | 1.5 |
| Alcohols | mass % | 4.8 |
| Others/Unknowns | mass % | 1.5 |

### EXAMPLE 2

A mixed alcohol feed having the feed composition shown in Table 3 was dehydrated into olefins over a gamma-alumina catalyst. The dehydration was completed at a reactor temperature of 337.8° C. (640° F.), at a reactor pressure of about 1034 kPa (g) (150 psig), and a weight-hourly-space velocity of about 1.0 hr⁻¹. The resulting non-water product distribution is shown in Table 4. In contrast to the reactor effluent produced with the catalyst in Example 1, the oxygenates and unconverted alcohols in this example were reduced to very low levels in the non-water reactor effluent, which simplifies the oxygenates removal process prior to light olefins separation. The reactor effluent produced with gamma alumina, once treated for oxygenates, would be suitable feed to the olefins separation process disclosed herein.

TABLE 3
Mixed Alcohol Feed Compositions

| | | |
|---|---|---|
| 2-Propanol | MASS-PCT | 1.2 |
| 1-Butanol | MASS-PCT | 0.1 |
| 1-Propanol | MASS-PCT | 18.1 |
| 2-Ethyl-1-hexanol | MASS-PCT | 0.7 |
| 2-methyl-1-propanol | MASS-PCT | 79.9 |

TABLE 4
Gamma Alumina - Normalized for Water

| | | |
|---|---|---|
| Paraffins | mass % | 0.5 |
| Ethylene | mass % | 0.0 |
| Propylene | mass % | 21.6 |
| 1-Butene&I-Butylene | mass % | 67.5 |
| Cis-2-butene | mass % | 4.7 |
| Trans-2-butene | mass % | 4.2 |
| Pentenes | mass % | 0.0 |
| Hexenes | mass % | 0.1 |
| Heptenes | mass % | 0.0 |
| C8 Olefins | mass % | 0.8 |
| C12 Olefins | mass % | 0.0 |
| Other Hydrocarbons | mass % | 0.0 |
| non-alcohol oxygenates | mass % | 0.1 |
| Alcohols | mass % | 0.4 |
| Others/Unknowns | mass % | 0.0 |

It should be appreciated and understood by those of ordinary skill in the art that various other components such as valves, pumps, filters, coolers, etc. were not shown in the drawings as it is believed that the specifics of same are well within the knowledge of those of ordinary skill in the art and a description of same is not necessary for practicing or understanding the embodiments of the present invention.

By the term “about,” we mean within 10% of the value, or within 5%, or within 1%.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

### Specific Embodiments

While the following is described in conjunction with specific embodiments, it will be understood that this description is intended to illustrate and not limit the scope of the preceding description and the appended claims.

A first embodiment of the invention is a process for providing a high purity olefin product, the process comprising dehydrating a feedstream comprising a mixture of alcohols having 3 to 8 carbon atoms and forming a mixed olefin stream and a water stream, the mixed olefin stream comprising a mixture of olefins having 3 to 8 carbon atoms, separating the mixed olefin stream into at least a C₃ olefin stream comprising olefins having 3 carbon atoms and a C₄₋₈ olefin stream comprising olefins having 4 to 8 carbon atoms, separating the C₄₋₈ olefin stream into a C₄ olefin stream comprising olefins having 4 carbon atoms and a C₅₋₈ olefin stream comprising olefins having 5 to 8 carbon atoms; and purifying at least one of the C₃ olefin stream and the C₄ olefin stream. An embodiment of the invention is one, any or all of prior embodiments in this paragraph up through the first embodiment in this paragraph wherein the mixed olefin stream further comprises oxygenated compounds, and further comprising removing the oxygenated compounds from the mixed olefin stream. An embodiment of the invention is one, any or all of prior embodiments in this paragraph up through the first embodiment in this paragraph wherein purifying the C₃ olefin stream comprises separating propane from the C₃ olefin stream to provide a purified C₃ olefin stream and a propane stream. An embodiment of the invention is one, any or all of prior embodiments in this paragraph up through the first embodiment in this paragraph wherein purifying the C₄ olefin stream comprises separating the C₄ olefin stream into at least an isobutylene stream and a normal butenes stream using a shape selective adsorbent. An embodiment of the invention is one, any or all of prior embodiments in this paragraph up through the first embodiment in this paragraph wherein purifying the C₄ olefin stream comprises separating the C₄ olefin stream into at least a 2-butene stream and further comprising introducing the 2-butene stream and an ethylene stream to a metathesis reaction zone to produce a propylene stream. An embodiment of the invention is one, any or all of prior embodiments in this paragraph up through the first embodiment in this paragraph wherein purifying the C₄ olefin stream comprises fractionating the C₄ olefin stream into an overhead stream comprising isobutylene and 1-butene and a bottoms stream comprising 2-butene, and further comprising separating the overhead stream into an isobutylene stream and a 1-butene stream using a shape selective adsorbent. An embodiment of the invention is one, any or all of prior embodiments in this paragraph up through the first embodiment in this paragraph wherein purifying the C₄ olefin stream comprises isomerizing the C₄ olefin stream to convert 1-butene to 2-butene; and fractionating the isomerized C₄ olefin stream into an isobutylene stream and a 2-butene stream. An embodiment of the invention is one, any or all of prior embodiments in this paragraph up through the first embodiment in this paragraph wherein purifying the C₄ olefin stream comprises converting the isobutylene and methanol to methyl tert-butyl ether in an etherification reaction zone to form an etherification effluent, separating the etherification effluent into a methyl tert-butyl ether stream and a stream comprising 1-butene and 2-butene, converting the methyl tert-butyl ether into isobutylene and methanol in a decomposition zone to form a decomposition effluent, and separating the decomposition effluent into an isobutylene stream and a methanol stream. An embodiment of the invention is one, any or all of prior embodiments in this paragraph up through the first embodiment in this paragraph further comprising fractionating the stream comprising 1-butene and 2-butene into a 1-butene stream and a 2-butene stream. An embodiment of the invention is one, any or all of prior embodiments in this paragraph up through the first embodiment in this paragraph further comprising separating the C₅₋₈ olefin stream into a C₅ olefin stream comprising olefins having 5 carbon atoms and a C₆₋₈ olefin stream comprising olefins having 6 to 8 carbon atoms. An embodiment of the invention is one, any or all of prior embodiments in this paragraph up through the first embodiment in this paragraph further comprising at least one of introducing the C₅ olefin stream and an ethylene stream to a metathesis reaction zone to produce a stream comprising propylene; and reforming the C₆₋₈ olefin stream to form an aromatics stream. An embodiment of the invention is one, any or all of prior embodiments in this paragraph up through the first embodiment in this paragraph further comprising cracking the C₄₋₈ olefin stream in an olefins cracking zone to produce a cracked ethylene stream, a cracked propylene stream, and a cracked C₄₋₈ stream, and wherein separating the C₄₋₈ stream comprises separating the cracked C₄₋₈ stream. The cracked C₄₋₈ stream is a mixed stream comprising olefins, paraffins, and aromatics.

A second embodiment of the invention is a process for providing a high purity olefin product, the process comprising dehydrating a feedstream comprising a mixture of alcohols having 3 to 8 carbon atoms and forming a mixed olefin stream and a water stream, the mixed olefin stream comprising a mixture of olefins having 3 to 8 carbon atoms, removing oxygenated compounds from the mixed olefin stream, separating the mixed olefin stream into at least a C₃ olefin stream comprising olefins having 3 carbon atoms and a C₄₋₈ olefin stream comprising olefins having 4 to 8 carbon atoms, separating the C₄₋₈ olefin stream into a C₄ olefin stream comprising olefins having 4 carbon atoms and a C₅₋₈ olefin stream comprising olefins having 5 to 8 carbon atoms, and purifying at least one of the C₃ olefin stream and the C₄ olefin stream. An embodiment of the invention is one, any or all of prior embodiments in this paragraph up through the second embodiment in this paragraph wherein purifying the C₃ olefin stream comprises separating propane from C₃ olefin stream to provide a purified C₃ olefin stream and a propane stream. An embodiment of the invention is one, any or all of prior embodiments in this paragraph up through the second embodiment in this paragraph wherein purifying the C₄ olefin stream comprises separating the C₄ olefin stream into at least an isobutylene stream and a normal butenes stream using a shape selective adsorbent. An embodiment of the invention is one, any or all of prior embodiments in this paragraph up through the second embodiment in this paragraph wherein purifying the C₄ olefin stream comprises separating the C₄ olefin stream into at least a 2-butene stream and further comprising introducing the 2-butene stream and an ethylene stream to a metathesis reaction zone to produce a propylene stream. An embodiment of the invention is one, any or all of prior embodiments in this paragraph up through the second embodiment in this paragraph wherein purifying the C₄ olefin stream comprises fractionating the C₄ olefin stream into an overhead stream comprising isobutylene and 1-butene and a bottoms stream comprising 2-butene, and further comprising separating the overhead stream into an isobutylene stream and a 1-butene stream using a shape selective adsorbent. An embodiment of the invention is one, any or all of prior embodiments in this paragraph up through the second embodiment in this paragraph wherein purifying the C₄ olefin stream comprises isomerizing the C₄ olefin stream to convert 1-butene to 2-butene; and fractionating the isomerized C₄ olefin stream into an isobutylene stream and a 2-butene stream. An embodiment of the invention is one, any or all of prior embodiments in this paragraph up through the second embodiment in this paragraph wherein purifying the C₄ olefin stream comprises converting the isobutylene and methanol to methyl tert-butyl ether in an etherification reaction zone to form an etherification effluent; separating the etherification effluent into a methyl tert-butyl ether stream and a stream comprising 1-butene and 2-butene; converting the methyl tert-butyl ether into isobutylene and methanol in a decomposition zone to form a decomposition effluent; and separating the decomposition effluent into an isobutylene stream and a methanol stream; and fractionating the stream comprising 1-butene and 2-butene into a 1-butene stream and a 2-butene stream. An embodiment of the invention is one, any or all of prior embodiments in this paragraph up through the second embodiment in this paragraph further comprising cracking the C₄₋₈ olefin stream in an olefins cracking zone to produce a cracked ethylene stream, a cracked propylene stream, and a cracked C₄₋₈ stream, and wherein separating the C₄₋₈ stream comprises separating the cracked C₄₋₈ stream. The cracked C₄₋₈ stream is a mixed stream comprising olefins, paraffins, and aromatics

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for providing a high purity olefin product, the process comprising:
dehydrating a feedstream comprising a mixture of alcohols having 3 to 8 carbon atoms and forming a mixed olefin stream and a water stream, the mixed olefin stream comprising a mixture of olefins having 3 to 8 carbon atoms;
separating the mixed olefin stream into at least a C₃ olefin stream comprising olefins having 3 carbon atoms and a C₄₋₈ olefin stream comprising olefins having 4 to 8 carbon atoms;
separating the C₄₋₈ olefin stream into a C₄ olefin stream comprising olefins having 4 carbon atoms and a C₅₋₈ olefin stream comprising olefins having 5 to 8 carbon atoms; and
purifying at least one of the C₃ olefin stream and the C₄ olefin stream.

2. The process of claim 1 wherein the mixed olefin stream further comprises oxygenated compounds, and further comprising removing the oxygenated compounds from the mixed olefin stream.

3. The process of claim 1 wherein purifying the C₃ olefin stream comprises separating propane from C₃ olefin stream to provide a purified C₃ olefin stream and a propane stream.

4. The process of claim 1 wherein purifying the C₄ olefin stream comprises separating the C₄ olefin stream into at least an isobutylene stream and a normal butenes stream using a shape selective adsorbent.

5. The process of claim 1 wherein purifying the C₄ olefin stream comprises separating the C₄ olefin stream into at least a 2-butene stream and further comprising:
introducing the 2-butene stream and an ethylene stream to a metathesis reaction zone to produce a propylene stream.

6. The process of claim 1 wherein purifying the C₄ olefin stream comprises fractionating the C₄ olefin stream into an overhead stream comprising isobutylene and 1-butene and a bottoms stream comprising 2-butene, and further comprising:
separating the overhead stream into an isobutylene stream and a 1-butene stream using a shape selective adsorbent.

7. The process of claim 1 wherein purifying the C₄ olefin stream comprises:
isomerizing the C₄ olefin stream to convert 1-butene to 2-butene; and
fractionating the isomerized C₄ olefin stream into an isobutylene stream and a 2-butene stream.

8. The process of claim 1 wherein purifying the C₄ olefin stream comprises:
converting the isobutylene and methanol to methyl tert-butyl ether in an etherification reaction zone to form an etherification effluent;
separating the etherification effluent into a methyl tert-butyl ether stream and a stream comprising 1-butene and 2-butene;
converting the methyl tert-butyl ether into isobutylene and methanol in a decomposition zone to form a decomposition effluent; and
separating the decomposition effluent into an isobutylene stream and a methanol stream.

9. The process of claim 8 further comprising:
fractionating the stream comprising 1-butene and 2-butene into a 1-butene stream and a 2-butene stream.

10. The process of claim 1 further comprising:
separating the C₅₋₈ olefin stream into a C₅ olefin stream comprising olefins having 5 carbon atoms and a C₆₋₈ olefin stream comprising olefins having 6 to 8 carbon atoms.

11. The process of claim 10 further comprising at least one of:
introducing the C₅ olefin stream and an ethylene stream to a metathesis reaction zone to produce a stream comprising propylene; and
reforming the C₆₋₈ olefin stream to form an aromatics stream.

12. The process of claim 1 further comprising cracking the C₄₋₈ olefin stream in an olefins cracking zone to produce a cracked ethylene stream, a cracked propylene stream, and a cracked C₄₋₈ stream, and wherein separating the C₄₋₈ stream comprises separating the cracked C₄₋₈ stream.

13. A process for providing a high purity olefin product, the process comprising:
dehydrating a feedstream comprising a mixture of alcohols having 3 to 8 carbon atoms and forming a mixed olefin stream and a water stream, the mixed olefin stream comprising a mixture of olefins having 3 to 8 carbon atoms;
removing oxygenated compounds from the mixed olefin stream;
separating the mixed olefin stream into at least a C₃ olefin stream comprising olefins having 3 carbon atoms and a C₄₋₈ olefin stream comprising olefins having 4 to 8 carbon atoms;
separating the C₄₋₈ olefin stream into a C₄ olefin stream comprising olefins having 4 carbon atoms and a C₅₋₈ olefin stream comprising olefins having 5 to 8 carbon atoms; and
purifying at least one of the C₃ olefin stream and the C₄ olefin stream.

14. The process of claim 13 wherein purifying the C₃ olefin stream comprises separating propane from C₃ olefin stream to provide a purified C₃ olefin stream and a propane stream.

15. The process of claim 13 wherein purifying the C₄ olefin stream comprises separating the C₄ olefin stream into at least an isobutylene stream and a normal butenes stream using a shape selective adsorbent.

16. The process of claim 13 wherein purifying the C₄ olefin stream comprises separating the C₄ olefin stream into at least a 2-butene stream and further comprising:
introducing the 2-butene stream and an ethylene stream to a metathesis reaction zone to produce a propylene stream.

17. The process of claim 13 wherein purifying the C₄ olefin stream comprises fractionating the C₄ olefin stream into an overhead stream comprising isobutylene and 1-butene and a bottoms stream comprising 2-butene, and further comprising:
separating the overhead stream into an isobutylene stream and a 1-butene stream using a shape selective adsorbent.

18. The process of claim 13 wherein purifying the C₄ olefin stream comprises:
isomerizing the C₄ olefin stream to convert 1-butene to 2-butene; and
fractionating the isomerized C₄ olefin stream into an isobutylene stream and a 2-butene stream.

19. The process of claim 13 wherein purifying the C₄ olefin stream comprises:
converting the isobutylene and methanol to methyl tert-butyl ether in an etherification reaction zone to form an etherification effluent;
separating the etherification effluent into a methyl tert-butyl ether stream and a stream comprising 1-butene and 2-butene;
converting the methyl tert-butyl ether into isobutylene and methanol in a decomposition zone to form a decomposition effluent; and
separating the decomposition effluent into an isobutylene stream and a methanol stream; and
fractionating the stream comprising 1-butene and 2-butene into a 1-butene stream and a 2-butene stream.

20. The process of claim 13 further comprising
cracking the C₄₋₈ olefin stream in an olefins cracking zone to produce a cracked ethylene stream, a cracked propylene stream, and a cracked C₄₋₈ stream, and wherein separating the C₄₋₈ stream comprises separating the cracked C₄₋₈ stream.
